(19) United States
(12) Patent Application Publication
(10) Pub. No.: US 2017/0057833 A1
(43) Pub. Date: Mar. 2, 2017

(54) RECIRCULATING TYPE ACTIVE SUBSTANCE TREATMENT SYSTEM

(71) Applicant: XYLEM IP HOLDINGS LLC., Rye Brook, NY (US)

(72) Inventors: David S. PALMER, Perry Green (GB); Robert M. ELLISON, Jamaica Plain, MA (US)

(21) Appl. No.: 15/254,425

(22) Filed: Sep. 1, 2016

Related U.S. Application Data
(60) Provisional application No. 62/212,692, filed on Sep. 1, 2015.

(51) Int. Cl. C02F 1/00; G01N 21/64; G01N 21/53; C02F 1/50; G01N 33/18; C02F 1/66; C02F 1/78; C02F 1/467; B63J 4/00; G01K 13/02

(52) U.S. Cl. CPC C02F 1/008; B63J 4/002; G01N 21/64; G01N 21/53; G01K 13/02; G01N 33/18; C02F 1/66; C02F 1/78; C02F 1/4674; C02F 1/50; C02F 2209/06; C02F 2209/02; C02F 2209/11; C02F 2209/006; C02F 2103/008

(57) ABSTRACT

A system is provided having a signal processor that receives signaling containing information about a quality of water being monitored for treatment in at least one ballast tank of a ballast treatment system and a compliance set point related to the quality of the water; and determines corresponding signaling containing information to recirculate and treat the water in the at least one ballast tank of the ballast treatment system until the quality of the water being monitored achieves the compliance set point, based upon the signaling received. The signal processor also provides the corresponding signaling as control signaling to recirculate and treat the water until the quality of the water being monitored achieves the compliance set point.

### CROSS-REFERENCE TO RELATED APPLICATIONS

This application claims benefit to provisional patent application Ser. No. 62/212,692 (911-020.002-2//X-SWI-0002), filed 1 Sep. 2015; which is incorporated by reference in its entirety.

### BACKGROUND OF THE INVENTION

1. Field of Invention

This invention relates to a technique for treating ballast water; and more particularly relates to a technique for technique for treating ballast water using a recirculation type active substance treatment system.

2. Description of Related Art

Ballast water quality may vary greatly according to where it is loaded, and factors that will affect the treatment level required include water temperature, pH and organic/solids content—a conventional treatment system using a calculated dosing level must take these variables into account with the probability of unnecessary inefficiency and over dosing in many cases.

Systems that treat during the ballasting/de-ballasting operation (often at high flow rates) are susceptible to being unable to achieve the required water quality compliance standards or cause delays to the ship because:
a. Conventional system designs have clearly defined maximum treatment dosing levels and flow rates and thus cannot compensate for very poor water quality or higher flow rates (as may be experienced during gravity load/discharge) without reducing the flow rate with a subsequent ship's operational delay implications that may be caused.
b. By law, ships must be able to ballast/de-ballast in 15 hours and this time limiting factor influences the size/flow rate of the ballast pumps that must be installed which in turn determines the size of the treatment plant required. High flow rate systems can be extremely expensive.
c. Any malfunction of the system during ballasting/de-ballasting will cause either—
   i. non-compliance issues due to having untreated water on board,
   ii. delay to the ship whilst repairs to the system are carried out,
   iii. delay to the ship if the system can still operate at reduced capacity/flow rate, and/or
   iv. potential environmental damage should the neutralization process malfunction.
d. The systems have only one opportunity to treat and achieve the required standards. The exception to this statement being that there are patent applications that states recirculation methods could be adopted as well as the main treatment process but this would increase the CAPEX of the system and is in effect fitting 2 systems. These secondary recirculation methods would also require large volumes of water to be recirculated to achieve adequate distribution throughout the tank plus, excessive pipework requirements if treated from one centrally located treatment plant.
e. Ships often ballast in tidal areas which can give varying incoming water qualities—the reaction time delay for the systems to adapt to these changes will likely cause either under or over dosing to occur.
f. Active substance type treatment system dosing levels are aimed at not causing a corrosive or damaging effect to the ballast water tank and coatings. Ports are often located in highly industrialized zones, and these dosing levels cannot take into consideration the quality (pH) of the ballast water being loaded. Once loaded and treated, the ballast water may be highly corrosive.
g. Neutralization (if required) is carried out during the de-ballasting operation at high flow rates with immediate overboard discharge. To perform this with no potentially adverse environmental consequences, the system must have the ability to dose precisely and mix correctly during changing flow rates and the challenging ballast stripping operation.

See also US 2014/0224714, which is assigned to the assignee of the present application and incorporated herein in its entirety by reference, which discloses a ballast treatment system.

In view of the aforementioned, there is a need in the industry for a better way to process ballast water for eventual discharge from a vessel, e.g., like a VLCC.

### SUMMARY OF THE INVENTION

By way of example, the present invention provides new and unique techniques for treating ballast water. In effect, the present invention provides an alternative ballast water treatment method that incorporates distinct process and component requirements in which all must be used to achieve the desired treatment made. There are known ballast water treatment, e.g., including that set forth above; however, none define a “stand alone” treatment method or the exact individual components necessary for implementing the present invention.

According to some embodiments, the present invention may include a system, e.g., in the form of a ballast water treatment system, comprising:

a signal processor or processing module configured to:
receive signaling containing information about a quality of water being monitored for treatment in at least one ballast tank of a ballast treatment system and a compliance set point related to the quality of the water; and
determine corresponding signaling containing information to recirculate and treat the water in the at least one ballast tank of the ballast treatment system until the quality of the water being monitored achieves the compliance set point, based upon the signaling received.

The apparatus may include one or more of the following additional features:

The signal processor or processing module may be configured to provide the corresponding signaling as control signaling to recirculate and treat the water until the quality of the water being monitored achieves the compliance set point.

The system may include a control system having the signal processor or processing module.

The control signaling may contain information to turn on/off a circulating pump and a submersible mixer configured in the at least one ballast tank of the ballast treatment system to recirculate and mix the water in the at least one ballast tank of the ballast treatment system until the quality of the water being monitored achieves the compliance set point.

The system may include the circulating pump and the submersible mixer, including where the submersible mixer provides a substantially complete distribution of a treatment agent in the water throughout the ballast.

The system may include, or take the form of, the ballast water treatment system having the at least one ballast tank.

The control signaling may contain information to turn on/off a combination of a neutralizing system and/or a disinfecting system to treat the water in the at least one ballast tank of the ballast treatment system until the quality of the water being monitored achieves the compliance set point.

The system may include the combination of the neutralizing system and/or the disinfecting system.

The at least one ballast tank may include multiple ballast tanks.

The system may be configured to process the ballast water in some combination of the multiple ballast tanks, e.g., including processing some part of the ballast water in either one ballast tank or multiple ballast tanks at a time.

The signal processor or processing module may be configured to:
receive respective signaling containing information about a respective quality of respective water being monitored for treatment in a respective ballast tank of the ballast treatment system and a respective compliance set point related to the quality of the water; and
determine respective corresponding signaling containing information to recirculate and treat the respective water in the respective ballast tank of the ballast treatment system until the quality of the respective water being monitored achieves the respective compliance set point, based upon the signaling received.

The control signaling may contain information to recirculate per hour only a small percentage of water in the at least one ballast tank, including where the small percentage if in a range of about 2% to 4%.

Part of the signaling may be received from a water quality monitor (WQM) configured to receive the water being monitored, sample the water, and provide monitoring signaling containing information about the quality of water being monitored for treatment in the at least one ballast tank of the ballast treatment system.

The system may include the water quality monitor.

The water quality monitor may include some combination of the following:
a. Variable Fluorometer to ensure VF treatment compliance has been achieved;
b. Total Residual Oxidant (TRO) Analyzer to ensure optimal oxidant and neutralization levels have been achieved;
c. pH sensor to compensate TRO measurements and track water quality conditions;
d. Temperature sensor to compensate other sensors and track water quality conditions;
e. fDOM sensor to compensate the VF sensor and track water quality conditions;
f. UV254 sensor to track water quality condition; and
g. Turbidity sensor to track water quality conditions and compensate the VF sensor.

Part of the signaling may be received from a memory device storing information about the compliance set point related to the quality of the water.

The compliance set point may be pre-programmed into the system, and/or adapted by the suitable ballast water treatment personnel on the ship/vessel. As one skilled in the art would appreciate, currently the compliance set point is determined by the International IMO D2 standard, and the United States Coast Guard (USCG) uses the same. However, several states in the US have brought out varying discharge limit guidelines for residual oxidant levels, e.g., which are more stringent. Based upon the a current understanding, e.g., in the industry, it is expected that other countries may follow in the future with their own guidelines. The pre-programmed compliance set point, and any adaptations thereto, would be made consistent with the appropriate standard.

By way of example, the signal processor or signal processor module may take the form of some combination of a signal processor and at least one memory including a computer program code, where the signal processor and at least one memory are configured to cause the system to implement the functionality of the present invention, e.g., to respond to signaling received and to determine the corresponding signaling, based upon the signaling received. Moreover, such a system may also include one or more of the features set forth above.

According to some embodiments, the present invention may include a method comprising steps for
receiving in a signal processor or processing module signaling containing information about a quality of water being monitored for treatment in at least one ballast tank of a ballast treatment system and a compliance set point related to the quality of the water; and
determining in the signal processor or processing module corresponding signaling containing information to recirculate and treat the water in the at least one ballast tank of the ballast treatment system until the quality of the water being monitored achieves the compliance set point, based upon the signaling received.
   The method may also include one or more of the features set forth above.

According to some embodiments, the present invention may include apparatus taking the form of
means for receiving in a signal processor or processing module signaling containing information about a quality of water being monitored for treatment in at least one ballast tank of a ballast treatment system and a compliance set point related to the quality of the water; and
means for determining in the signal processor or processing module corresponding signaling containing information to recirculate and treat the water in the at least one ballast tank of the ballast treatment system until the quality of the water being monitored achieves the compliance set point, based upon the signaling received.
   Such apparatus may also include one or more of the features set forth above.

According to some embodiments of the present invention, the apparatus may also take the form of a computer-readable storage medium having computer-executable components for performing the steps of the aforementioned method. The computer-readable storage medium may also include one or more of the features set forth above.

In summary, the key differential of the present invention and the prior techniques known in the art is that present invention does not rely on the application of theoretical dosing levels to achieve compliance. Instead, the present invention relies wholly upon water quality monitoring equipment to control the treatment/neutralizing dose levels to the exact minimum amount required dependent on the water quality to achieve compliance. This desired level of treatment accuracy and efficiency can be achieved by the present invention process described. In contrast, the known time limiting factor during ballasting/de-ballasting operations and known treating at high flow rates directly into the ballast water as previously set forth above does not allow this and therefore the treatment process according to the present invention may be undertaken and implemented at any other time (in port, at sea, at anchor) after the ballast water tanks have been filled.

### BRIEF DESCRIPTION OF THE DRAWING

The drawing includes FIGS. 1-5, which are not necessarily drawn to scale, and include the following:

FIG. 1 shows a diagram of a system overview of a ballast water treatment system, according to some embodiments of the present invention.

FIG. 2 shows a diagram of a process logic flow, e.g., for implementing a system like that shown in FIG. 1, according to some embodiments of the present invention.

FIG. 3 shows a typical 300,000 Dead Weight Tonnage (DWT) Very Large Crude Carrier (VLCC).

FIG. 4 shows an example of a feasibility study (e.g., treat time only) conducted in relation to the present invention.

FIG. 5 shows a block diagram of a system, e.g., having a signal processor or signal processing module, for implementing signal processing functionality, according to some embodiments of the present invention.

### DETAILED DESCRIPTION OF BEST MODE OF THE INVENTION

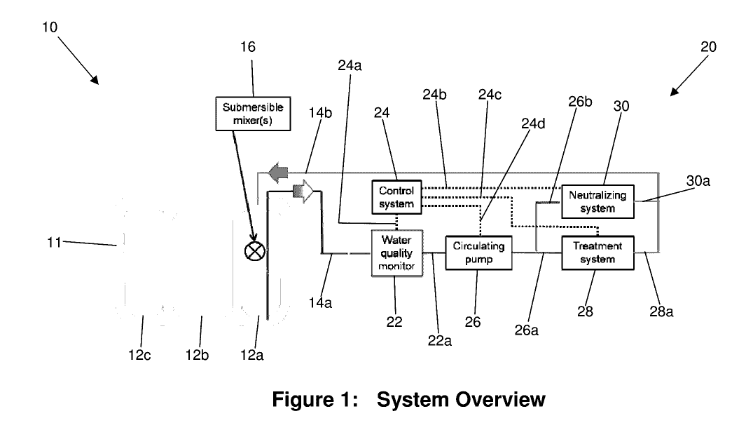

### FIG. 1

### A System Overview

By way of example, FIG. 1 shows a system generally indicated as 10 for implementing a recirculation type active substance treatment system, according to some embodiments of the present invention. The system 10 includes a VLCC 11 in combination with an active substance treatment system generally indicated by 20.

The VLCC 11 includes one or more ballast tanks 12a, 12b and 12c configured therein for holding ballast tank water and fluidically coupled to the active substance treatment system 20 via piping 14a, 14b. The VLCC 11 includes one or more submersible mixer(s), e.g., one of which is indicated by reference numeral 16, for mixing the ballast tank water in the one or more ballast tanks 12a, 12b and 12c. A VLCC like element 11 having ballast tanks like element 12a, 12b and 12c is known in the art; and the scope of the invention is not intended to be limited to any particular type or kind thereof either now known or later developed in the future. The active substance treatment system 20 is also understood to be arranged or configured in the VLCC 11 and in relation the one or more ballast tanks 12a, 12b and 12c; and the scope of the invention is not intended to be limited to any particular arrangement or configuration thereof either now known or later developed in the future.

The active substance treatment system 20 includes a water quality monitor 22, a control system 24, a circulation pump 26, a treatment system 28 and a neutralizing system 30. The circulation pump 26, the treatment system 28 and the neutralizing system 30 are all known in the art; and the scope of the invention is not intended to be limited to any particular type or kind thereof either now known or later developed in the future. The water treatment system 20 receives the ballast water from the one or more ballast tanks 12a, 12b and 12c via the piping 14a, which is provided to the water quality monitor 22. The control system 24 provides suitable control signaling along control signaling lines indicated by lead lines and reference numerals 24a, 24b, 24c, 24d to the water quality monitor 22, the circulation pump 26, the treatment system 28 and the neutralizing system 30, e.g., as shown. The control system 24 may be configured to determine suitable control signaling, e.g., consistent with that set forth herein. Piping 22a couples together the water quality monitor 22 and the circulating pump 26; piping 26a, 26b couples together the circulating pump 26, the treatment system 28 and the neutralizing system 30; and piping 28a, 30a couples together the treatment system 28, the neutralizing system 30 and the piping 14b, which provides treated and neutralized ballast water back to the one or more ballast tanks 12a, 12b, 12c. By way of example, the piping 14a, 14b, 22a, 26a, 26b, 28a and 30a may be configured with suitable valves for receiving suitable valve control signaling, e.g., from the control system 24, to provide/pump the ballast water to and from the ballast tanks 12a, 12b and 12c and the water treatment system 20, as well as between the water quality monitor 22, the treatment system 28 and the neutralizing system 30, as would be appreciated by one skilled in the art.

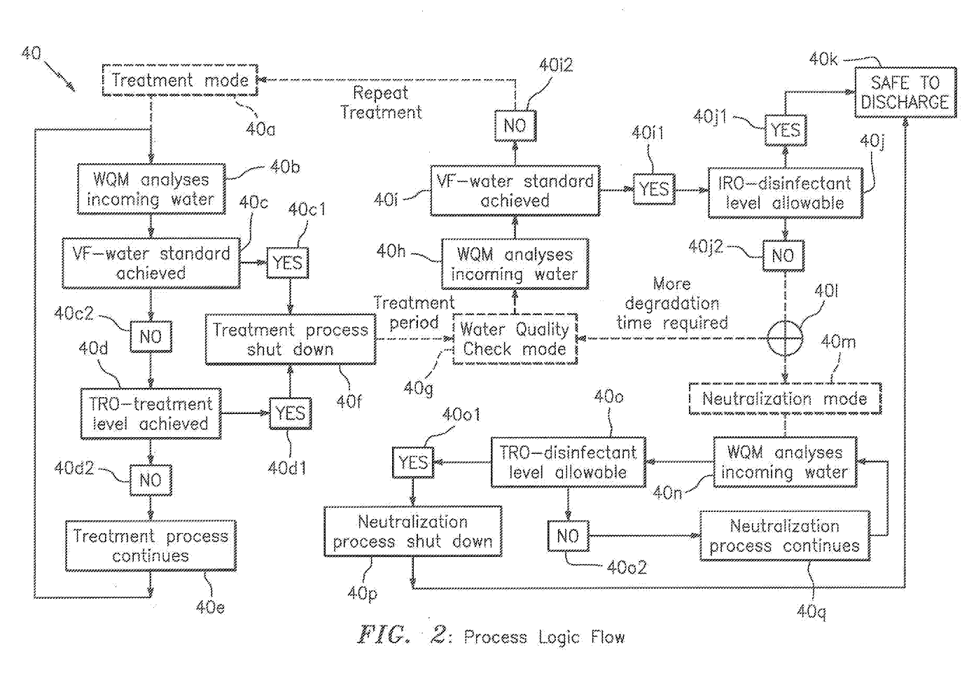

### FIG. 2

### Process Logic Flow Chart

FIG. 2 shows a process logic flow generally indicated as 40, e.g., for implementing by the control system 24 in the system 10 shown in FIG. 1, according to some embodiments of the present invention. The control system 24 may be configured to implement steps in the process logic flow 40, including steps 40a through 40q, as shown, for providing the ballast water treatment, as follows:

### The Treatment Mode 40a

In step 40a, the control system 24 may be configured to implement a treatment mode, e.g., that may include performing treatment mode steps of the process logic flow 40 for a first time, or repeating the treatment mode steps of the process logic flow 40 after performing the treatment mode of step 40a and determining in step 40i and 40i2 that the VF water standard was not achieved. In either case, the treatment mode of step 40a include the following steps:

In step 40b, the control system 24 may be configured to signal the water quality monitor 22 via control signal along line 24a to analyze incoming ballast water, e.g., using a variable fluorescence sensing device (VF) and/or a total residual oxidant (TRO) sensing device configured or arranged in the water quality monitor 22.

In steps 40c, 40c1, if the VF-water standard is achieved, then the control system 42 implements step 40f to shut the treatment process down.

In steps 40c, 40c2, if the VF-water standard is not achieved, then the control system 42 implements step 40d.

In steps 40d, 40d1, if the TRO-treatment level is achieved, then the control system 24 implements step 40f to shut the treatment process down.

In steps 40d, 40d2, if the TRO-treatment level is not achieved, then the control system 24 implements step 40e to continue the treatment process, e.g., by returning to step 40b for repeating the aforementioned treatment mode steps 40b through 40f.

In step 40f, the control system 24 shuts down the treatment process for a treatment period, e.g., which is determined consistent with that set forth herein.

### The Water Quality Check Mode 40g, after the Treatment Process Shut Down and Treatment Period

In step 40g, after shutting down the treatment process for the treatment period, the control system 24 may be configured to implement a water quality check mode, which may include implementing one or more further water treatment control loops, e.g., having one or more of the steps 40h through 40q. For example, the control system 24 may be configured to implement steps 40h through 40j and analyze the VF standard and TRO level, and depending on a determination if more degradation time may be required, then implement a neutralization mode in steps 40m through 40q. Depending on the implementation of these steps, the control system 24 based upon these determination implements step 40k when the ballast water is safe to discharge.

For example, in step 40h, the control system signals the WQM 22 to analyze incoming ballast water.

In steps 40i and 40i1, if the VF-water standard is achieved, then the control system 24 implement step 40j.

In steps 40i and 40i2, if the VF-water standard is not achieved, then the control system 24 repeats the treatment, and returns to step 40a for repeating the treatment mode step.

In steps 40j and 40j1, if the TRO-disinfectant level is allowable, then the control system 24 implements step 40k based upon a determination that the ballast water is safe to discharge.

In steps 40j, 40j2, if the TRO-treatment level is not allowable, then the control system 24 implements step 40l, and if more degradation time may be required, then the control system 24 implements and repeats the water quality check mode in step 40g.

If the control system 24 determines that more degradation time may not be required, then the control system 24 implements step 40m and initiates a neutralization mode.

In step 40n, the control system 24 signals the WQM 22 to analyze the incoming ballast water.

In steps 40o and 40o1, if the TRO-disinfectant level is allowable, then the control system 24 implements step 40p to shut the neutralization process down and implements step 40k based upon a determination that the ballast water is safe to discharge.

In step 40o, 40o2, if the TRO-disinfectant level is not allowable, then the control system 24 implements step 40q to continue the neutralization process, e.g., by returning to step 40n for repeating this neutralization steps.

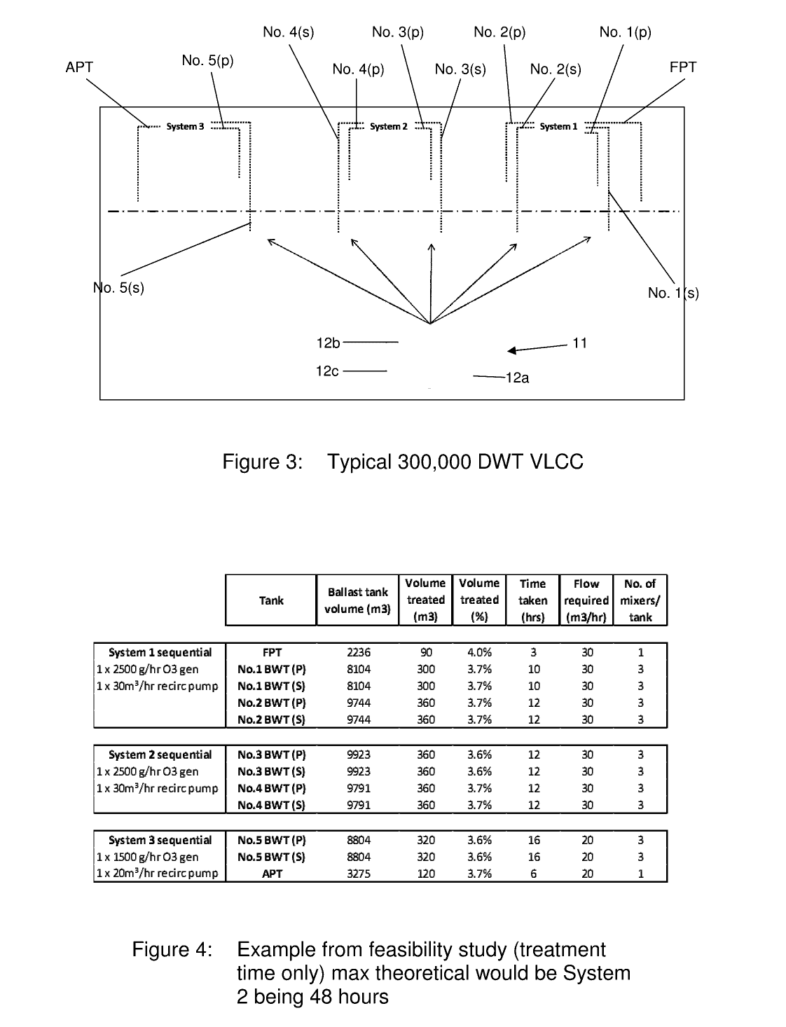

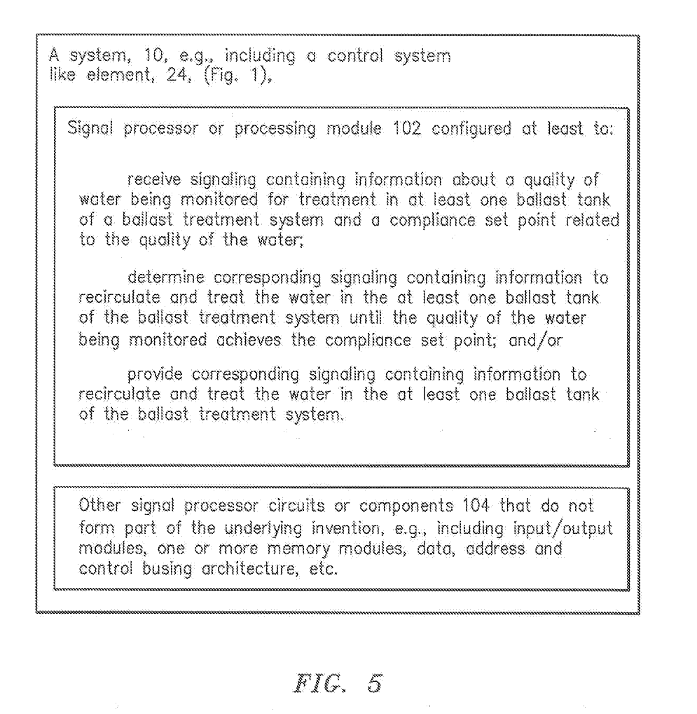

### FIGS. 3-4

By way of example, FIG. 3 shows the typical 300,000 DWT VLCC, e.g., which is generally indicated by 11 consistent with that set forth above. The VLCC 11 includes three ballast systems (Nos. 1, 2 and 3), each configured on starboard and port sides of the VLCC. By way of example, ballast system no. 1 includes five (5) ballast tanks FPT, No. 1(s), No. 1(p), No. 2(s), No. 2(p); Ballast system no. 2 includes four (4) ballast tanks No. 3(s), No. 3(p), No. 4(s), No. 4(p); and Ballast system no. 3 includes five (5) ballast tanks No. 4(s), No. 4(p), No. 5(s), No. 5(p), APT.

By way of example, FIG. 4 shows the results of a feasibility study base upon an implementation according to the present invention for a VLCC like element 11.

### FIG. 5

### Implementation of Signal Processing Functionality

By way of example, FIG. 5 shows a system 10 according to some embodiments of the present invention for implementing the associated signal processing functionality.

The system 10 may include a control system like element 24 (FIG. 1) having a signal processor or processing module 102 configured at least to:
receive signaling containing information about a quality of water being monitored for treatment in at least one ballast tank of a ballast treatment system and a compliance set point related to the quality of the water; and
determine corresponding signaling containing information to recirculate and treat the water in the at least one ballast tank of the ballast treatment system until the quality of the water being monitored achieves the compliance set point, based upon the signaling received.

In operation, the signal processor or processing module 102 may also be configured to provide the corresponding signaling, e.g., in the form of control signaling, to recirculate and treat the water in the at least one ballast tank of the ballast treatment system until the quality of the water being monitored achieves the compliance set point, consistent with that set forth herein. The control signal may be provided to one or more of the water quality monitor 22, the circulating pump 26, the treatment system 28 and the neutralizing system 30.

The signal processor or processing module 102 may be configured in, or form part of, a system having a control system or controller like the control system 24 (FIG. 1), e.g., for providing control signaling along lines 24a through 24d as shown in FIG. 1.

By way of example, the signaling received may be generated and provided by the water quality monitor 22 (FIG. 1).

By way of example, the functionality of the signal processor or processing module 102 may be implemented using hardware, software, firmware, or a combination thereof. In a typical software implementation, the signal processor or processing module 102 would include one or more microprocessor-based architectures having, e. g., at least one signal processor or microprocessor like element 102. One skilled in the art would be able to program with suitable program code such a microcontroller-based, or microprocessor-based, implementation to perform the signal processing functionality disclosed herein without undue experimentation. For example, the signal processor or processing module 102 may be configured, e.g., by one skilled in the art without undue experimentation, to receive the signaling containing information about a quality of water being monitored for treatment in at least one ballast tank of a ballast treatment system and a compliance set point related to the quality of the water, consistent with that disclosed herein.

Moreover, the signal processor or processing module 102 may be configured, e.g., by one skilled in the art without undue experimentation, to determine the corresponding signaling containing information to recirculate and treat the water in the at least one ballast tank of the ballast treatment system until the quality of the water being monitored achieves the compliance set point, consistent with that disclosed herein.

Moreover still, the signal processor or processing module 102 may be configured, e.g., by one skilled in the art without undue experimentation, to provide the corresponding signaling containing information, e.g., as control signaling, to recirculate and treat the water in the at least one ballast tank of the ballast treatment system until the quality of the water being monitored achieves the compliance set point, consistent with that disclosed herein.

The scope of the invention is not intended to be limited to any particular implementation using technology either now known or later developed in the future. The scope of the invention is intended to include implementing the functionality of the signal processor(s) 102 as stand-alone processor, signal processor, or signal processor module, as well as separate processor or processor modules, as well as some combination thereof.

The signal processor or processing module 102 may also include, e.g., other signal processor circuits or components 102, including random access memory or memory module (RAM) and/or read only memory (ROM), input/output devices and control, and data and address buses connecting the same, and/or at least one input processor and at least one output processor, e.g., which would be appreciate by one skilled in the art.

### Key Process Benefits of the Present Invention

1) The use of in-tank mixers like element 16 in the pre-loaded ballast water offers uniform and stable water quality conditions throughout the treatment process and ensures complete distribution of the treatment throughout the tank contents, which provides overall process uniformity and stability.
2) During treatment and neutralization, the process times are not fixed, and may only run until compliance set points are achieved, which provides process efficiency.
3) The use of the in-tank mixers like element 16 may offer further benefits by agitating and maintaining the sediment in the one or more ballast tanks 12a, 12b, 12c in suspension—
   a. During the disinfecting and neutralizing processes, the active treatment agents will be in contact with all organisms/cells/bacteria that may normally be protected by the sediment, which provides process efficiency.
   b. Use of the mixers like element 16 prior to ballast discharge may reduce the build-up of sediment in the one or more ballast tanks 12a, 12b, 12c maintaining the cargo carrying capacity of the ship. Moreover, the sediment that may be discharged overboard will have been treated, which provides benefit to the ship owner re meeting environmental requirements in any given port of call.
4) A very low and consistent recirculation flow rate (5-30 m³/hr. depending on system size) may be used that allows for accurate process requirements to be achieved, which provides process accuracy.
5) The disinfecting and neutralizing treatment levels may be gradually increased in a controlled manner until the required level and compliance are achieved without the possibility of under or over dosing, which provides for process efficiency.
6) The process according to the present invention only requires a small % of the tank contents to be recirculated (e.g., about 2%-4%), which provides for process efficiency.
7) The process accuracy and efficiency offers a reduction in overall treatment power/fuel consumption requirement, which provides for an overall OPEX reduction.
8) The present invention offers the ability to monitor and provide an alert to the potential corrosive effect of the ballast water. Potentially, the neutralizing process may be further adapted to counteract potential corrosion/coating damage issues, which provide ship owner benefit in reducing overall maintenance costs.
9) The extended process duration allows for smaller process equipment to be used, which provides for overall CAPEX reduction.
10) The use of smaller equipment offers a large reduction in power requirement whilst the processes are running negating a potential lack of available on-board power, which provides for ship operational benefit like reducing the cost associated with provisioning the overall system power requirements for the ship.
11) The flexibility of the system design may allow for multiple smaller treatment units to be employed (treatment plants and mixers) in lieu of larger treatment units (offering redundancy in the event of malfunction but, with a slightly increased CAPEX and longer process duration if running at reduced capacity), which provides an overall reliability benefit.
12) The present invention has the ability to utilize the 3 most common forms of active substance treatment—Ozone, Electro Chlorination and Chemical

Addition, which provides for process flexibility.

### Key Differentials and Benefits of the Present Invention

1) The present invention may be configured to achieve the required compliance standards, e.g., regardless of the ballast water quality taken on board.
2) To achieve the treatment accuracy and efficiency, the present invention may be configured to utilize a water quality monitor that incorporates a more comprehensive array of sensors, e.g. including—
   a. Variable Fluorometer to ensure treatment compliance has been achieved,
   b. Total Residual Oxidant (TRO) Analyzer to ensure optimal oxidant and neutralization levels have been achieved,
   c. pH sensor to compensate TRO measurements and track water quality conditions pH/ORP sensors to compensate,
   d. Temperature sensor to compensate other sensors and track water quality conditions,
   e. fDOM sensor to compensate the VF sensor and track water quality conditions,
   f. UV254 sensor to track water quality condition, and
   g. Turbidity sensor to track water quality conditions and compensate the VF sensor.
3) To achieve the process efficiency during treatment and neutralization, the present invention may be configured to utilize a highly efficient way for injecting the disinfecting and neutralizing liquids into the water stream created by the in-tank mixers like element 16.
4) To achieve the process efficiency during treatment and neutralization, the present invention may be configured to utilize a wide range of in-tank mixing equipment (e.g., types and sizes). The present invention also allows for more flexibility in relation to the location and sizing of the mixers to ensure that all contents of the ballast water tank (all shapes and volumes) are thoroughly mixed in the most efficient and economic method.
5) The sizing of the system is also more flexible and not governed by the flow rate capability of the installed ballast water pumps. The system may be sized according to the operational requirements of the ship as determined by the ship owner—for short voyage times larger systems offering faster process times or conversely, for long voyage times smaller systems could be used. There will be CAPEX implications for smaller/larger systems and OPEX implications for shorter/longer process run times but, the ship owner has the ability to take these variables into consideration, e.g., when using the present invention for ballast water treatment.
6) The present invention may be more readily implemented and adapted to respond to changes imposed in the future, e.g., including future environmental requirements. For example, should compliance standards alter; the system (VF and TRO compliance set points) need only be re-programmed to increase/decrease disinfection/neutralization dosing level requirements. No new equipment and/or infrastructure would likely be required.

### System Design Factors

The system design may be configured in a highly flexible way and may be tailored to the ship owner's operational requirements for the ship/vessel. Required shorter or longer treatment times may be used to determine system factors such as number of treatment systems installed, size of treatment/neutralization systems, recirculation pump flow rates, number and size of mixers installed per tank etc.

To ensure the maximum treatment efficiency of the system, the ship may be configured or installed with a known commercially readily available ballast water filter (e.g., after the main ballast pumps) that need only be used during the loading of the ballast water. By way of example, the ballast water filter may be chosen to offer a mesh size of no greater than 40 microns.

### Process and Operation Description

The system functions in 3 separate semi-automatic operational modes and the logic flow diagram illustrates the complete process—

Control System

The complete system may be configured to operate from commands given to the control system 24, e.g. by the ship's operating staff. The key command functions may include—
Tank selection if the system 10 is configured to treat/neutralize multiple tanks,
Process selection—treatment/water quality check/neutralization,
Start process, and
Manual override—shut down running process.
   During operation of the selected process, the Control System 24 may be configured to operate fully automatically until the process is complete. The Control System 24 may be configured to make available to ship's operating staff the following information—
The Control system 24 may be configured to constantly provide information regarding when the Treatment, Water Quality Check or Neutralizing process has been completed, and the treatment process has shut down.
The Control system 24 may be configured to constantly provide information regarding current process status, and the status of all the ballast tanks on board.
The Control System 24 may be configured to have data logging capabilities providing historic time/date information for the following—
   The dates, times, types and kinds of ballast water treatment process applied to each ballast tank,
   The recorded treatment effectiveness prior to discharge (VF−Fv/Fm reading),
   The recorded water quality prior to discharge (oxidant potential measurement), and
   If required, data could be logged for all other Water Quality Monitor sensors.
The Control system 24 may be configured to monitor all system components for correct function whilst running and have the ability to alert operating staff and automatically safely shut down the system 20 in the event of component failure or system malfunction.
The Control System 24 may be configured to alert operating staff that the water quality poses a threat of accelerated corrosion and or potential coating damage within the ballast water tank.

All process modes may be started by the ship's operating staff after selecting the required ballast tank to be treated, e.g., by providing one or more user inputs that may be received by the control system 24. Prior to starting, the control system 24 may be configured to verify that the appropriate tank valves are in the correct position. the control system 24 may be configured to receive and process the one or more user inputs provided by the ship's operating staff, and adapt or change the control system processing routine or steps based upon the same.

### 1. Treatment Mode

By way of example, the ballast water may be taken from the ballast tank 12a, 12b, 12c using a low flow recirculating pump like element 26 (FIG. 1) and passed through the treatment process whereby the water becomes a concentrated disinfecting liquid (the treatment process may use Ozone, Electro Chlorination or Chemical Addition as a means of generating the disinfectant—active substances).

The concentrated disinfecting liquid may be then returned to the ballast tank 12a, 12b, 12c where submersible mixing equipment like element 16 distributes the concentrated liquid throughout the entire tank contents. The number of mixers and injection points may vary according to the ballast tank shape, capacity and ship operational requirements (see system design factors). The control system may be configured to run the mixers 16 continuously throughout the treatment process, as well as to run the mixers 16 periodically and intermittently as needed.

The Water Quality Monitor 22 (FIG. 1) may be configured to constantly or periodically monitor the treatment process. The treatment process may be configured to run until the disinfection concentration in the ballast water tank 12a, 12b, 12c has been raised to predetermined levels that are known to effectively treat ballast water to required standards under test conditions, or that the water quality is such that no further treatment is required. This function avoids unnecessary over dosing and optimizes overall system efficiency.

When the required treatment level has been achieved, the Control System 24 may be configured to advise the ship's operating staff that the process is complete and has been shut down.

1(a) Treatment Period

As one skilled in the art would appreciate, active substance treatments are not instantaneous and the full disinfection effect may take from minutes to several hours. The time that must be allowed for full disinfection to take place will vary according to various factors, e.g., such as treatment type, water temperature and water quality. The treatment period may not or may not be pre-programmed. In addition, the Water Quality Monitor 22 may incorporate one or more required sensors to assess the above variables and recommend a minimum treatment period. The selection and determination of any particular treatment period may be adaptive, e.g., based upon the further processing of sensed and recorded temporal and historical data related to the same

1(b) Treatment Degradation Time

As one skilled in the art would appreciate, over time water has the ability to naturally consume and degrade the disinfection. Again, factors such as treatment type, water temperature and water quality may influence the time required for the water to recover to a quality such that it may be discharged directly into the environment without causing any adverse effects. The Water Quality Monitor 22 may be configured to incorporate one or more required sensors to assess the above variables and recommend a minimum degradation time. The selection and determination of any particular degradation time may be adaptive, e.g., based upon the further processing of sensed and recorded temporal and historical data related to the same

### 2. Water Quality Check Mode

As one skilled in the art would appreciate, the treatment period time must be allowed for a full disinfection process to take place; however, the degradation time allowed can be decided by the ship's operating staff based upon recommendations from the Water Quality Monitor 22 or the operational requirements of the ship (voyage time).

The control system 24 may be configured to run the treatment system in the “water quality check” mode (see step 40g (FIG. 2)), whereby the tank mixer(s) like element 16 may be started and ran for a predetermined short period. The control system 24 may be configured to shut down the mixer(s) like element 16, and/or to start and run the recirculating pump like element 26 to allow the Water Quality Monitor like element 22 to assess the ballast water quality. The WQM 22 may be configured to assess the treatment effectiveness and the disinfection degradation—this assessment will advise the ship's operating staff if the treatment process is satisfactory and the water is to compliance standards or, if not, further treatment may be required. If the treatment process is determined to be satisfactory, then the WQM 22 may be configured to check and advise if the water quality is suitable for immediate discharge. If not suitable, the ballast water should be allowed further degradation time (see step 40l) or run through the neutralization process like that implemented re steps 40m through 40q if the degradation time is not available prior to discharge.

### 3. Neutralization Mode

In the neutralization mode in steps 40m through 40q (FIG. 2), the ballast water may be taken from the ballast tank 12a, 12b, 12c using the low flow recirculating pump like element 26 and passed through a neutralization process whereby the required neutralizing substance may be mixed with the ballast water to form a concentrated neutralizing liquid.

The concentrated neutralizing liquid may then be returned to the ballast tank 12a, 12b, 12c where the submersible mixing equipment like element 16 may be configured to distribute the concentrated liquid throughout the entire tank contents. The mixers 16 may be configured to run continuously throughout the neutralizing process, as well as run periodically and intermittently as needed.

The neutralizing process may also be constantly monitored by the Water Quality Monitor 22 and the process may run until the water quality in the ballast water tank 12a, 12b, 12c is suitable for discharge via step 40k. By way of example, the Control System 24 may be configured to then advise the ship's operating staff that the process is complete and has been shut down.

### Safety

For safety reasons, the system 10 may typically not necessarily by configured to have the ability to automatically sequentially treat/neutralize multiple tanks, although the scope of the invention is not intended to be limited to the same. For example, embodiments are envisioned, and the scope of the invention is intended to include, implementing a fully automated system, including one that can be adapted remotely, turned on/off remotely, e.g., including via a centralized control center using a satellite-based, or global positioning based, wireless signaling exchange.

Each individual system may be configured to incorporate flow and pressure sensors on the recirculating water lines that in the event of pipe failure shall shut down the system and advise the operator. Loss of ballast tank capacity due to pipe failure should not present ship stability issues due to the low recirculating flow rate and shut down of the associated tank valves. Tank level monitoring may also be incorporated if not already fitted.

By way of example, different treatment (Ozone, Electro Chlorination, and Chemical Addition) types may be available and implemented for use with this overall system process, which all have their individual requirements in terms of safe process control and, due to the wide variances; these will not be covered under this disclosure.

### The Compliance Set Point

According to some embodiments, and as would be appreciated by one skilled in the art, the compliance set point may be a preprogrammed compliance set point, a user-defined compliance set point, as well as an adaptive compliance set point.

By way of example, the preprogrammed compliance set point may form part of a library of preprogrammed compliance set point stored in a database in the system 11, e.g., that may be selected as a user input by the ship's operating staff.

By way of further example, the user-defined compliance set point may be provided by the ship's operating staff, as a preprogrammed compliance set point, or as an override to a preprogrammed compliance set point, or a compliance set point currently being used.

By way of still further example, the adaptive compliance set point may be adapted, e.g. based upon sensed and/or recorded temporal or historical data that depends on numerous factors set forth herein, as well as tolerances or parameters provided by the ship's operating staff, consistent with that set forth herein. The adaptive compliance set point may also be adapted or determined by implementing an adaptive compliance set point algorithm that takes into account one or more of various parameters and sensed data set forth herein, including the ship's voyage time, environmental requirements at the port of call, the current quality of the ballast water, etc.

The scope of the invention is not intended to be limited to the type or kind of compliance set point used, and is intended to include compliance set points, and techniques for determining compliance set points, e.g., that are now known or later developed in the future.

### THE SCOPE OF THE INVENTION

While the invention has been described with reference to an exemplary embodiment, it will be understood by those skilled in the art that various changes may be made and equivalents may be substituted for elements thereof without departing from the scope of the invention. In addition, may modifications may be made to adapt a particular situation or material to the teachings of the invention without departing from the essential scope thereof. Therefore, it is intended that the invention not be limited to the particular embodiment(s) disclosed herein as the best mode contemplated for carrying out this invention.

What is claimed is:

1. A system comprising:
a signal processor or processing module configured to:
receive signaling containing information about a quality of water being monitored for treatment in at least one ballast tank of a ballast treatment system and a compliance set point related to the quality of the water; and
determine corresponding signaling containing information to recirculate and treat the water in the at least one ballast tank of the ballast treatment system until the quality of the water being monitored achieves the compliance set point, based upon the signaling received.

2. A system according to claim 1, wherein the signal processor or processing module is configured to provide the corresponding signaling as control signaling to recirculate and treat the water until the quality of the water being monitored achieves the compliance set point.

3. A system according to claim 2, wherein the control signaling contains information to turn on/off a circulating pump and a submersible mixer configured in the at least one ballast tank of the ballast treatment system to recirculate and mix the water in the at least one ballast tank of the ballast treatment system until the quality of the water being monitored achieves the compliance set point.

4. A system according to claim 3, wherein the system comprises the circulating pump and the submersible mixer, including where the submersible mixer provides a substantially complete distribution of a treatment agent in the water throughout the ballast.

5. A system according to claim 3, wherein the system comprises, or take the form of, a ballast water treatment system having the at least one ballast tank.

6. A system according to claim 2, wherein the control signaling contains information to turn on/off a combination of a neutralizing system and/or a disinfecting system to treat the water in the at least one ballast tank of the ballast treatment system until the quality of the water being monitored achieves the compliance set point.

7. A system according to claim 6, wherein the system comprises the combination of the neutralizing system and/or the disinfecting system.

8. A system according to claim 3, wherein the at least one ballast tank comprises multiple ballast tanks.

9. A system according to claim 8, wherein the signal processor or processing module configured to:
receive respective signaling containing information about a respective quality of respective water being monitored for treatment in a respective ballast tank of the ballast treatment system and a respective compliance set point related to the quality of the water; and
determine respective corresponding signaling containing information to recirculate and treat the respective water in the respective ballast tank of the ballast treatment system until the quality of the respective water being monitored achieves the respective compliance set point.

10. A system according to claim 2, wherein the control signaling containing information to recirculate per hour only a small percentage of water in the at least one ballast tank, including where the small percentage if in a range of about 2% to 4%.

11. A system according to claim 1, wherein part of the signaling is received from a water quality monitor configured to receive the water being monitored, sample the water, and provide monitoring signaling containing information about the quality of water being monitored for treatment in the at least one ballast tank of the ballast treatment system.

12. A system according to claim 11, wherein the system comprises the water quality monitor.

13. A system according to claim 11, wherein the water quality monitor comprises some combination of the following:
a. Variable Fluorometer to ensure treatment compliance has been achieved;
b. Total Residual Oxidant (TRO) Analyzer to ensure optimal oxidant and neutralization levels have been achieved;
c. pH sensor to compensate TRO measurements and track water quality conditions;
d. Temperature sensor to compensate other sensors and track water quality conditions;
e. fDOM sensor to compensate the VF sensor and track water quality conditions;
f. UV254 sensor to track water quality condition; and
g. Turbidity sensor to track water quality conditions and compensate the VF sensor.

14. A system according to claim 1, wherein part of the signaling is received from a memory device storing information about the compliance set point related to the quality of the water.
